(19) United States
(12) Patent Application Publication
(10) Pub. No.: US 2022/0096695 A1
(43) Pub. Date: Mar. 31, 2022

(54) COMPOSITION FOR DIFFUSING VOLATILE SUBSTANCES

(71) Applicant: ZOBELE HOLDING SPA, TRENTO (IT)

(72) Inventors: Joaquim LLORENTE ALONSO, Barcelona (ES); Alba GRAUS FERRER, Barcelona (ES); Moisés CABALLERO TAPIA, Barcelona (ES)

(21) Appl. No.: 17/280,265

(22) PCT Filed: Sep. 26, 2019
(86) PCT No.: PCT/EP2019/076039

§ 371 Date: Mar. 26, 2021

(30) Foreign Application Priority Data
Sep. 26, 2018 (ES) P201830927

(51) Int. Cl. A61L 9/012; A61L 9/03; A01N 25/10

(52) U.S. Cl. CPC A61L 9/012; A01N 25/10; A61L 9/037

(57) ABSTRACT

A composition for diffusing volatile substances, comprising wax, which further comprises a solid material that includes volatile substances, said solid material being in the form of particles, and which has a volatile substance absorption capacity greater than the volatile substance absorption capacity of the wax. It enables a composition for diffusing volatile substances to be obtained which enables the percentage of volatile substances to be increased, that is, the percentage of fragrance, and the release time to be lengthened.

The present invention relates to a composition for diffusing volatile substances, in particular, for use as an air freshener or insecticide.

### BACKGROUND OF THE INVENTION

There are different products on the market that contain impregnated wax-based volatile substance formulations for use in a heated device, without needing to use combustion to release the volatile substances. The wax in these containers melts and releases the volatile substances into the environment.

Current solutions are limited in their properties as a product for diffusing volatile substances due to the limitations of the wax itself.

The wax is characterised by having a relatively low absorption by weight limit of formulation depending on the wax used. For example, wax of only paraffinic origin does not support more than 3% by weight and it is not recommended for other waxes to have much more than 6% by weight.

When trying to increase the fragrance ratio above this threshold, the wax begins to expel the fragrance creating an effect where it seems that the product “sweats”. As a result, the ratio of product size to the amount of fragrance is limited.

Another feature of this type of product is that the wax, once it is heated and goes into a liquid state, is not able to retain volatile substances for a long time, causing this type of product to be perceived very intensely at the beginning with a high degree of fragrance evaporation, but within a few hours there is no longer a fragrance.

Therefore, an objective of the present invention is to provide a composition for diffusing volatile substances that enables the percentage of volatile substances to be increased, that is, the percentage of fragrance, and the release time to be lengthened.

### DESCRIPTION OF THE INVENTION

The aforementioned drawbacks are resolved by the composition of the invention, having other advantages that are described below.

The composition for diffusing volatile substances, according to the present invention, comprises wax, and it is characterised in that it further comprises solid material that includes volatile substances.

Advantageously, said solid material is in the form of particles, for example, of ethylene-vinyl acetate (EVA).

Furthermore, in the composition for diffusing volatile substances according to the present invention, said solid material has a volatile substance absorption capacity greater than the volatile substance absorption capacity of the wax.

For example, the volatile substance absorption capacity of the solid material is twice that of the volatile substance absorption capacity of the wax.

According to a preferred embodiment, the composition for diffusing volatile substances of the present invention comprises a percentage by weight of wax between 60 and 70%, for example, 65%, and a percentage by weight of solid material between 40 and 30%, for example, 35%.

If desired, the wax can also comprise volatile substances, although it is also possible for the volatile substances to only be in the solid material, in which case, the volatile substances of the solid material may be different from the volatile substances of the wax.

The solid material used is capable of absorbing more formula than the wax (>6%). When the wax melts, it goes into a liquid state, but the material remains solid.

The main advantages of the composition according to the present invention are the following:
The initial volume of the conventional product is maintained with only wax, but the percentage of fragrance is increased, depending on the percentage amount of wax or impregnated solid particles of the final composition.
By having a greater amount of volatile substances, the user perceives a greater intensity of fragrance, and faster.
The solid particles retain volatile substances inside the same for longer when the composition is heated. This enables the life of the product to be extended compared to one that only contains wax.

### BRIEF DESCRIPTION OF THE DRAWINGS

In order to better understand the foregoing, drawings are provided wherein, schematically and solely by way of non-limiting example, a practical case of embodiment is represented.

FIG. 1 is a graph showing the weight loss percentage over time of a composition according to the present invention and a comparison composition.

### DESCRIPTION OF A PREFERRED EMBODIMENT

The composition for diffusing volatile substances according to the present invention comprises wax and solid material, for example, solid particles.

Said solid material is preferably ethylene-vinyl acetate (EVA) and has a capacity to absorb more volatile substances than wax, such as for example, twice the volatile substances absorption capacity than that of wax, considering the same volume.

In the composition according to the present invention, the volatile substances may be contained in the wax and in the solid material, or only in the solid material, as desired. In the event that the wax and solid material include volatile substances, they can be different from each other to provide different features.

An example of the composition according to the present invention and a comparative example of a conventional composition are described below.

In this example, the composition was a 11 g block of wax, with 5% of volatile substances, that is, 0.55 g.

It was evaluated how much solid material (EVA) would be needed to double the amount of volatile substances in the same volume as the original block. After an initial estimate and considering that said solid material is capable of absorbing 20% of volatile substances, a mixture is obtained as a result (considering that both materials had a similar density) where approximately 65% by weight was wax impregnated with volatile substances and 35% by weight was EVA impregnated with volatile substances, obtaining twice the amount of volatile substances for the same occupied volume.

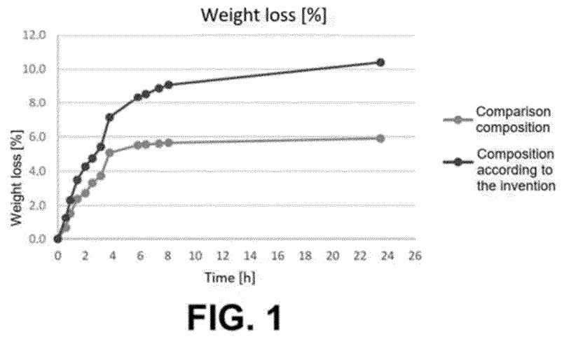

A comparison composition was also made with only wax impregnated with volatile substances and a mixture with the percentages mentioned above. A heated device was used and the two products were compared. After 24 hours, the results are shown in the graph of FIG. 1 and in the attached table.

| | | Composition |
|---|---|---|
| | Comparison | according to |
| | composition | the invention |
| Initial Weight (grams) | 11.1 | 10.8 |
| Evaporated volatile substances (grams) | 0.65 | 1.13 |
| T (° C.) | 83 | 83 |

Based on said graph and said table, the following conclusions can be drawn:
The amount of evaporated volatile substances is much greater for the same evaporation time and, therefore, user perception is also greater.
After 5 hours, the amount of evaporated volatile substances in the wax alone is practically level, while in the example according to the present invention, the evaporated amount continues to increase beyond 8 hours, the solid material helping to slow evaporation.
As seen in the final evaporation, not only are there more volatile substances at the beginning, but the composition according to the invention is capable of evaporating the volatile substances an additional 74% after 24 hours.

Despite the fact that reference has been made to a specific embodiment of the invention, it is evident for a person skilled in the art that the described composition is susceptible to numerous variations and modifications, and that all the aforementioned details may be replaced by other, technically equivalent ones, without falling outside the scope of protection defined by the attached claims.

1. A composition for diffusing volatile substances, comprising:
wax; and
a solid material that includes volatile substances.

2. The composition for diffusing volatile substances according to claim 1, wherein said solid material is in the form of particles.

3. The composition for diffusing volatile substances according to claim 1, wherein said solid material has a volatile substance absorption capacity greater than the volatile substance absorption capacity of the wax.

4. The composition for diffusing volatile substances according to claim 1, wherein a percentage by weight of wax is between 60 and 70% and a percentage by weight of solid material is between 40 and 30%.

5. The composition for diffusing volatile substances according to claim 3, wherein a first volatile substance absorption capacity of the solid material is twice that of a second volatile substance absorption capacity of the wax.

6. The composition for diffusing volatile substances according to claim 1, wherein the wax further comprises wax volatile substances, the volatile substances of the solid material being different from the wax volatile substances of the wax.

7. The composition for diffusing volatile substances according to claim 1, wherein only the solid material comprises volatile substances.

8. The composition for diffusing volatile substances according to claim 1, wherein said solid material is ethylene-vinyl acetate (EVA).

9. The composition for diffusing volatile substances according to claim 2, wherein said solid material is in the form of particles.

10. The composition for diffusing volatile substances according to claim 9, wherein a percentage by weight of wax is between 60 and 70% and a percentage by weight of solid material is between 40 and 30%.

11. The composition for diffusing volatile substances according to claim 2, wherein a percentage by weight of wax is between 60 and 70% and a percentage by weight of solid material is between 40 and 30%.

12. The composition for diffusing volatile substances according to claim 3, wherein a percentage by weight of wax is between 60 and 70% and a percentage by weight of solid material is between 40 and 30%.

13. The composition for diffusing volatile substances according to claim 2, wherein said solid material is ethylene-vinyl acetate (EVA).

14. The composition for diffusing volatile substances according to claim 3, wherein said solid material is ethylene-vinyl acetate (EVA).

15. The composition for diffusing volatile substances according to claim 9, wherein said solid material is ethylene-vinyl acetate (EVA).

16. The composition for diffusing volatile substances according to claim 4, wherein said solid material is ethylene-vinyl acetate (EVA).

17. The composition for diffusing volatile substances according to claim 10, wherein said solid material is ethylene-vinyl acetate (EVA).

18. The composition for diffusing volatile substances according to claim 11, wherein said solid material is ethylene-vinyl acetate (EVA).

19. The composition for diffusing volatile substances according to claim 12, wherein said solid material is ethylene-vinyl acetate (EVA).

20. The composition for diffusing volatile substances according to claim 5, wherein said solid material is ethylene-vinyl acetate (EVA).

21. The composition for diffusing volatile substances according to claim 6, wherein said solid material is ethylene-vinyl acetate (EVA).

22. The composition for diffusing volatile substances according to claim 7, wherein said solid material is ethylene-vinyl acetate (EVA).
